(19) United States
(12) Patent Application Publication
(10) Pub. No.: US 2010/0261967 A1
(43) Pub. Date: Oct. 14, 2010

(54) VIDEO LARYNGOSCOPE SYSTEM AND DEVICES

(75) Inventors: John Allen Pacey, Vancouver (CA); Mitchell Visser, Burnaby (CA); Reza Ahmadian Yazdi, Richmond (CA); Yongkook Kim, Burnaby (CA)

Correspondence Address: BLACK LOWE & GRAHAM, PLLC, 701 FIFTH AVENUE, SUITE 4800, SEATTLE, WA, 98104, US

(73) Assignee: Verathon Inc., Bothell, WA (US)

(21) Appl. No.: 12/760,519

(22) Filed: Apr. 14, 2010

Related U.S. Application Data
(60) Provisional application No. 61/319,835, filed on Mar. 31, 2010.
(60) Provisional application No. 61/169,246, filed on Apr. 14, 2009.
(60) Provisional application No. 61/261,739, filed on Nov. 16, 2009.

(51) Int. Cl. A61B 1/267

(52) U.S. Cl. 600/186; 600/188

(57) ABSTRACT

An embodiment encompassing reusable video laryngoscopes that are insertable into transparent or non-transparent disposable sheaths having clear optically clear viewing windows that receive contact with camera lens. The reusable video laryngoscopes are configured to be detachably secured by locking tabs located in the transparent or non-transparent disposable sheaths with complementary shaped posts and ledges of the video laryngoscopes. The video laryngoscopes that are secured into the transparent or non-transparent disposable sheaths may be inserted into the patient's mouth to provide clear camera viewing through the optically clear window ports of the transparent or non-transparent disposable sheaths to allow endotracheal procedures to be undertaken. The transparent or non-transparent disposable sheaths are sterilizable and may be used just once for a given patient. The video laryngoscopes in the form of a baton are detachably removeable from the disposable sheath exposed to a patient.

### CROSS REFERENCES TO RELATED APPLICATIONS

The application claims priority to and incorporates by reference in their entirety U.S. Provisional Patent Applications Nos. 61/319,835 filed Mar. 31, 2010; 61/261,739 filed Nov. 16, 2009; and 61/169,446 filed Apr. 14, 2009. This application also incorporates by reference in their entirety U.S. Pat. No. 6,142,144 filed Apr. 1, 1998 and its U.S. Provisional Patent Application Nos. 60/074,355 filed Feb. 10, 1998 and 60/067,205 filed Dec. 7, 1997; U.S. Pat. No. 6,543,447 filed Dec. 6, 2000 and its U.S. patent application Ser. No. 09/704,507 filed Nov. 2, 2007 and 09/060,891 filed Apr. 15, 1998; and U.S. Pat. No. 6,655,377 filed Jan. 30, 2003 and its U.S. patent application Ser. No. 09/732,129 filed Dec. 6, 2000 and 09/704,507 filed Nov. 2, 2000, and U.S. Provisional Patent Application Nos. 60/352,283 filed Jan. 30, 2002; 60/223,330 filed Aug. 7, 2000; 60/168,711 filed Dec. 6, 1999; 60/074,355 filed Feb. 10, 1998, and 60/067,205 filed Dec. 1, 1997. All patents and patent applications are incorporated by reference in their entirety.

### FIELD OF THE INVENTION

Disclosure herein is generally directed to the field of airway management and examination of the upper airway, and, in particular, to apparatuses that permit examination of the upper airway and/or intubation.

### BACKGROUND OF THE INVENTION

Endotracheal intubation provides the current preferred method for control of the airway for mechanical ventilation. The process involves passing an endotracheal tube (ETT) through the mouth, past the tongue, and to and through the vocal cords and larynx to seal the airway. This protects the patency of the airway and protects it from aspiration of gastric contents, foreign substances, or secretions. The complex and invasive procedure occurs regularly in surgery and emergency departments throughout the word. It is increasingly performed in pre-hospital settings such as ambulances, medical evacuation helicopters, and by military medics in combat and near-combat situations. It is well known that failure to intubate when required can lead to death or serious injury. Intubation is a complex process which presents numerous challenges, as well as myriad possible injuries to the patient short of death from de-oxygenation. In all instances, the better the view which the instrument of choice provides to the intubator, the lower the likelihood of error resulting in injury or death. Traditional laryngoscopes relied on opening the upper airway to allow a direct line of sight from the intubator's eye to the larynx. Subsequent developments in laryngoscopes utilized fiberoptic bundles, sometimes coupled to video displays. More recently, laryngoscopes with video cameras have made it possible to display the image of the airway anatomy from a position beyond the teeth, and in some instances allow the intubator to identify the relevant anatomical landmarks without repositioning the patient.

### BRIEF DESCRIPTION OF THE DRAWINGS

Preferred and alternative examples of the present invention are described in detail below with reference to the following drawings:

FIG. 1 depicts a substantially side view of a video laryngoscope system 10 having a single-use transparent sterilizeable sheath configured to receive a detachable video baton stabilized by the video baton's side posts that are engageable with the side-securing tabs of the sheath;

FIG. 2 depicts the detachable video baton during insertion into the sterilizeable sheath in which the baton's side posts slidably engage the side-securing tabs and shown to be transiting midway across the sloping services of sheath's side-securing tabs;

FIG. 3 depicts the finished insertion movement of the video baton into the sheath in which the baton's side posts have transited across the tab's sloping surfaces and held in the rounded apex of a concave V-shaped cradle and the inward slope of the side-securing tab;

FIG. 4 depicts an enlargement of the baton side post and sheath side-securing tab depicted in FIG. 3;

FIG. 5 depicts the sheath open end with the video baton completely engaged and secured within the V-shaped cradle of the sheath;

FIG. 6 depicts a substantially side view of a video laryngoscope system 50 having a single-use transparent sterilizeable sheath configured to receive a detachable video baton stabilized with the video baton's side ledges and rearward lip respectively engaged with the internal side supports and rearward locking tab of the sheath;

FIG. 7 depicts a substantially rear perspective view of the video laryngoscope system 50 showing the sheath aperture in which the internal side supports are located on the walls of the baton handle chamber that is continuous with a sheath brim on which is mounted the locking tab to respectively engage the baton handle's ledges and lip;

FIG. 8 depicts a rearward perspective view of the detachable video baton of FIG. 6 during insertion into the sterilizeable sheath;

FIG. 9 depicts a sideward perspective view of a portion of the detachable video baton of FIG. 6 during insertion into the sterilizeable sheath;

FIG. 10 depicts a rearward perspective view of the detachable video baton of FIG. 6 inserted and secured into the sterilizeable sheath;

FIG. 11 depicts a sideward perspective view of the portion of the detachable video baton of FIG. 6 inserted and secured into the sterilizeable sheath;

FIGS. 12A-G depict constructional plane and cross sectional views of an embodiment of the sheath depicted in FIG. 1;

FIGS. 13A-G depict constructional plane and cross sectional views of another embodiment of the sheath depicted in FIG. 1;

FIGS. 14A-E depict constructional plane and cross sectional views of an embodiment of the sheath depicted in FIG. 6;

FIGS. 15A-E depict constructional plane and cross sectional views of another embodiment of the sheath depicted in FIG. 6;

FIGS. 16A-D depict a series of substantially side and perspective view of a video laryngoscope system 100 having a single-use non-transparent sterilizeable sheath configured to receive a detachable video baton stabilized with the video baton's side posts that are engageable with opposing side-securing tabs located along the edges of the V-shape cradle of an alternate embodiment of the sheath depicted in FIG. 1;

FIGS. 17A-C depict a series of substantially perspective views and a partial cross section view of the sheath in a video laryngoscope system 200 having a non-transparent sheath of as an alternated embodiment to the transparent sheath depicted in FIG. 1;

FIGS. 18A-C depict a substantially side perspective views of a video laryngoscope system 300 having a single-use non-transparent sterilizeable sheath configured to receive a detachable video baton stabilized with the video baton's side ledges and rearward lip respectively engaged with the internal side supports and sideward locking tab of an alternate embodiment of the sheath depicted in FIG. 6;

FIGS. 19A-C depicts substantially side perspective views of a video laryngoscope system 400 having a single-use non-transparent sterilizeable sheath as an alternate embodiment of the transparent sheath depicted in FIG. 1, the non-transparent sheath configured to receive a detachable video baton stabilized with the video baton's side posts that are engageable with the side-securing tabs of the sheath;

FIGS. 20A-B depict laryngoscope placement and advancement in a patient's mouth;

FIG. 21 depicts engagement of the vallecula by the tip of the laryngoscope to move the epiglottis upward to reveal the glottic aperture;

FIG. 22 depicts direct lifting of the epiglottis by the tip of the laryngoscope to move the epiglottis upward to reveal the glottic aperture;

FIG. 23 depicts a stylet adjacent to an ETT;

FIGS. 24A and B depicts placement of the ETT via the stylet in the oropharynx of the patient;

FIG. 25 depicts a patient cross section showing side views of the ETT/Stylet in position for passing through the glottic aperture;

FIGS. 26-29 illustrate the passing the ETT in the patient with the laryngoscope system and device embodiments; and

FIG. 30 depicts a flowchart of algorithm for using the system and device embodiments to position the ETT within a patient's airway.

### DETAILED DESCRIPTION OF THE PARTICULAR EMBODIMENTS

Several embodiments of a video-based intubation laryngoscope and system are described that allow for examination of the upper airway and intubation. The system employs video laryngoscope embodiments configured to view a patient's glottis, reposition the patient's epiglottis, view the glottic aperture and convey video images of the patient's upper airway anatomy including the glottis and/or glottic aperture and surrounding area to a video monitor viewable by the laryngoscope user. An endotracheal tube (ETT) is placed within the patient's oral cavity and its position relative to the patient's GA is noted from the images presented on the video monitor. Based on video images displayed on the monitor, the ETT is advanced into the trachea through the patient's glottic aperture via an accessory stylet located within the lumen of the ETT.

Embodiments of the laryngoscope include substantially clear housings or sheaths intended for single use into which a video baton is inserted. The video baton includes a video camera and a light source and is brought distally against an optical window located on the posterior side of the blade of the laryngoscope. Images obtained from the video baton are conveyed to a video monitor viewable by the laryngoscope user. The blade is used to reposition the epiglottis by engagement of the patient's vallecula, or alternatively, directly lifting the epiglottis to reveal the glottic aperture. An ETT loaded with a stylet is inserted into the mouth under direct vision and advanced until the tip of the ETT appears in the video monitor image, at or near the distal portion of the laryngoscope blade and proximal to the glottic aperture. Viewing the monitor, the ETT is then advanced forward through the glottic aperture into the patient's trachea, while the stylet is removed.

Improved intubation speed and intubation accuracy is accomplished by the specific configurations in that unobstructed, real time or “live” views are immediately obtained on a viewable display monitor. The laryngoscope's configurations provide an aiming aid that allows real time re-positioning of the video laryngoscope to optimally align the tip of the ETT with the glottic aperture just prior to advancing the ETT through the glottic aperture from the video laryngoscope. The video laryngoscopes provide clear, direct images of the larynx, vocal cords, and laryngeal area on the display monitor and offer a means to control the trajectory of the ETT toward and through the glottic aperture.

The laryngoscope includes a handle, a blade configured to engage the epiglottis to reveal the glottic aperture which is visualized by the video camera and lighting unit located on the posterior side of the laryngoscope blade, directed towards the distal end. The video-based laryngoscope embodiments may be made with materials that allow for sterilization and re-use, or alternatively, be configured to include a disposable portion which houses a removable video camera and lighting member located within an internal chamber. The removable video camera and lighting member unit is sufficiently sealed within the internal chamber to prevent moisture or fluids from reaching the internal optical electronics of the video camera and lighting member. In yet another embodiment a disposable laryngoscope adapter or sheath may be detachably affixable to the laryngoscope equipped with the non-removable camera to provide a clean to sterile surface which permits re-use of the video-based laryngoscope without having to undergo washing or decontamination procedures.

An exemplary embodiment described below includes an apparatus having a laryngoscope housing or sheath that is configured to detachably receive a video camera unit. The laryngoscope sheath has a handle and a blade with an internal chamber that spans from the handle and terminates with an optically clear window on the posterior side of the blade directed toward the distal end. The internal chamber is configured to receive an insertable video camera unit and secure the video unit within the internal chamber. The video unit includes a video camera and a light source to illuminate an anatomical region within the field of view of the lens.

Retaining clasps or tabs located on the internal walls of the chamber near the handle portion detachably engage against the surfaces of the video camera such that the lens portion of the video camera unit may be positioned against the internal side of the optically clear window. The chamber is designed to receive the video camera unit and to firmly place the lens of the video unit near or against the interior side of the window by engaging the retaining clasp with the surface features or structures of the video unit. In another exemplary embodiment of the video camera laryngoscope housing or sheath may have its non-window portions such as the handle, blade, and the internal chamber may be transparent, translucent, or opaque. In yet further embodiments, the window is also a lens that focuses the camera. In some embodiments, the retaining clasps are configured, such as by tapers or by eccentric positioning with respect to the mating handle components, to press the distal end of the video baton and the camera firmly against the window, thereby minimizing the presence of air and/or moisture in the space between the camera and window. Airspace and attendant moisture could result in fogging of the camera. Additionally, if the window is also a focusing lens, the positioning of the camera relative to the lens is important for image quality.

The laryngoscope housing or sheath may be constructed of materials for single use and thus disposable, or made with materials amenable to cleaning, decontamination, and/or sterilization and thus be reusable or configured for multi-use with other insertable video laryngoscopes that are configured to be detachably secured by the locking tabs located in the interior portions of the disposable sheath.

The video laryngoscopes secured into the transparent or non-transparent disposable sheaths may be inserted into the patient's mouth to provide clear camera viewing through the optically clear ports of the transparent or non-transparent disposable sheaths to allow endotracheal procedures to be undertaken. The transparent or non-transparent disposable sheaths are sterilizable and may be used just once for a given patient. The video laryngoscopes in the form of a baton are configured to be detachably removable from the disposable sheath after removal from a patient and re-inserted into another sterilize disposable sheath for insertion into another patient. Alternatively, reusable sheaths undergo high-level disinfection after a clinical exam has been completed. In other embodiments, the sheath's intended single-use can be enforced by constructing the retaining clasps or tabs in a frangible configuration such that upon removal or detachment of an inserted video baton from the sheath's internal chamber, the frangible tabs break off so that they become unavailable to secure the video baton within the sheath's chamber.

Embodiments described encompass reusable video laryngoscopes that are insertable into transparent or non-transparent disposable sheaths having clear optically clear viewing windows that receive contact with camera lens. The reusable video laryngoscopes are configured to be detachably secured by locking tabs located in the transparent or non-transparent disposable sheaths with complementary shaped posts and ledges of the video laryngoscopes. The video laryngoscopes that are secured into the transparent or non-transparent disposable sheaths may be inserted into the patient's mouth to provide clear camera viewing through the optically clear window ports of the transparent or non-transparent disposable sheaths to allow endotracheal procedures to be undertaken. The transparent or non-transparent disposable sheaths are pre-sterilized and may be used just once for a given patient. The video laryngoscopes in the form of a baton are configured to be detachably removable from the disposable sheaths and removed from a patient and re-inserted into another sterilized disposable sheath for insertion into another patient. The reusable video laryngoscope batons may then undergo high-level disinfection after a series of clinical exams have been completed.

Other embodiments described herein include a video laryngoscope system having a video laryngoscope having a handle and a transparent sheath having a blade with an optically clear window, a chamber configured to receive the handle and the camera, and a locking tab configured to engage at least one surface of the laryngoscope handle. The configuration provides for insertion of video laryngoscope into the chamber of the sheath or stat such that the video laryngoscope fits into the chamber with the locking tab to attachably engage with the at least one surface to slidably bring the camera in contact with the optically clear window. Other embodiments provide for the at least one locking tab to include adjoining tapered wedges that are engageable with the surface of the at least one surface of the laryngoscope handle. The video laryngoscopes in the form of a baton are detachably removable from the disposable sheath after use and can be readily inserted into another sterile disposable sheath for application to a different patient.

Cameras of the video batons may include CCD or CMOS configurations that may be placed at a point of angulation of the blade of the disposable sheaths near the midpoint to provide for advantageous positioning of the camera at some distance from the glottic opening to allow a degree of perspective and wide angle viewing.

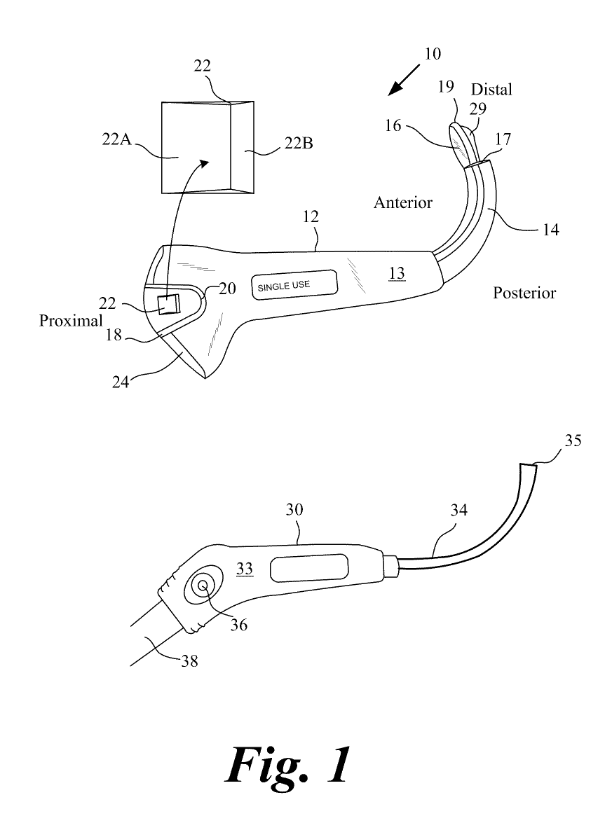

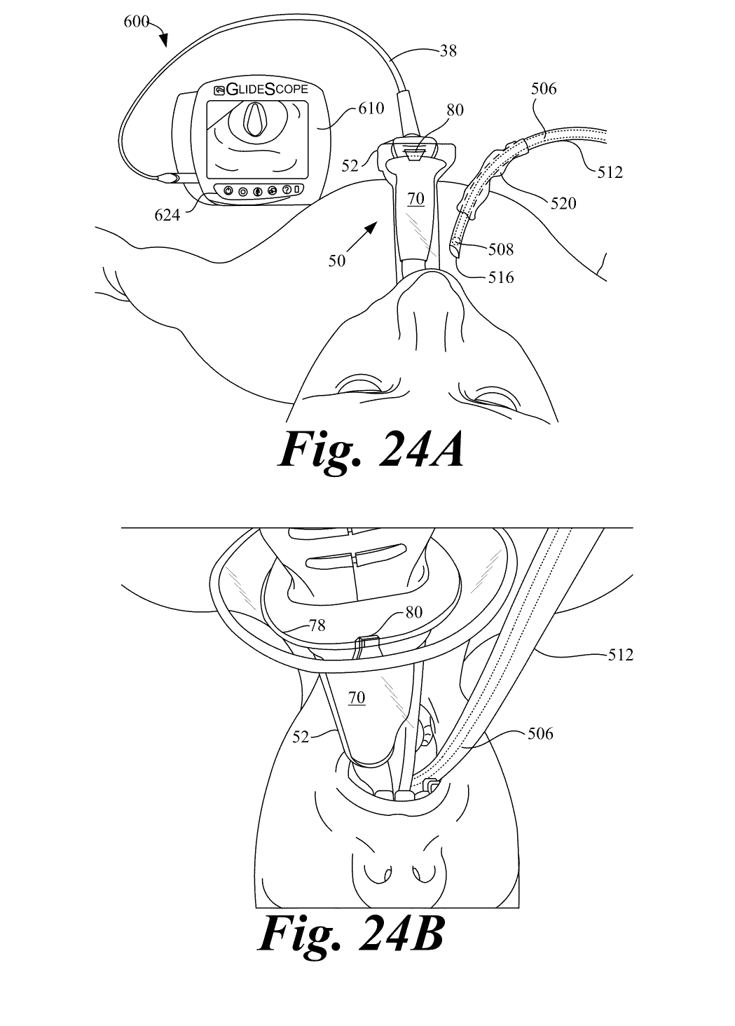

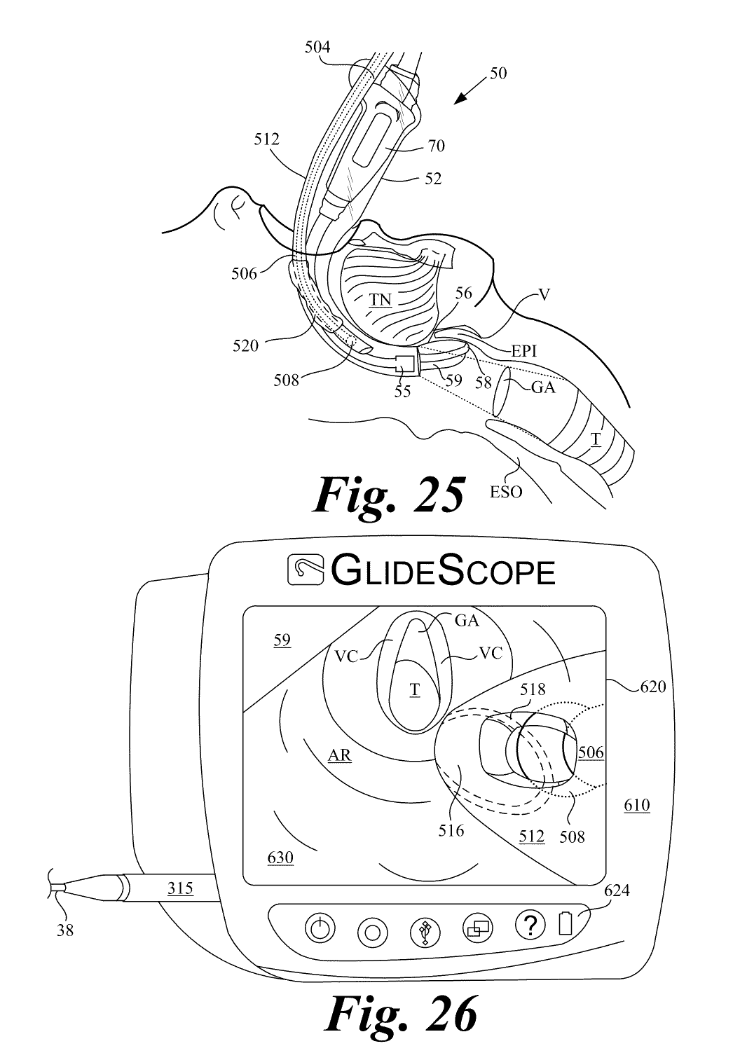

FIG. 1 depicts a substantially side and partially perspective view of a video laryngoscope 10 having a sterilizeable sheath 12 configured to receive a detachable video baton 30 internally placeable and detachably secureable within the sterilizeable sheath 12. The video baton 30 includes a non-flexible handle 33, a flexible cable 34 extending from the handle 33, and a video camera and lighting member 35 extending from the cable 34 and located at the distal end of the video baton 30. Extending from the proximal side of the handle 33 is a power and video cable 38 that is connectable with an analog or digital video monitor 610 shown in FIGS. 24A and 26 below. The video baton 30 is intended for multiple uses with individual sterilizeable sheaths 12, where each individual sheath 12 is intended for single-use events in a patient. The sterilizable sheath 12 may be transparent or opaque and includes a sheath handle 13 defining a chamber 24 similarly shaped to and slightly larger than the video baton 30. Sheath 12 includes two concave V-shaped cradles 18 having a rounded apex 20. The V-shaped cradles 18 are disposed diametrically opposite each other as shown in FIG. 5 below. Located between the rounded apex 20 and the opening of the V-shaped cradle 18 is a side-securing or locking tab 22. The locking tab 22 resides on the internal surfaces of the sterilizable sheath 12.

The sheath 12 has a proximal end with chamber 24 opening for receiving the video baton 30. The anterior portion includes the proximal blade portion 14 and distal blade portion 16. The distal blade portion terminates with distal tip 19 for lifting the epiglottis or for engaging the vallecula V of a patient to lift the epiglottis EPI to reveal the glottic aperture GA. Window 17 is positioned on the posterior side of the sheath 12 and video camera and lighting member 35 of the video baton 30 terminate at video window 17 and are directed towards the distal end when inserted.

The V-shaped cradle 18 is convex shaped and includes a rounded apex 20. In the magnified insert, a locking tab 22 is shown to include two adjoining wedge surfaces 22A and 22B. Wedge surface 22A faces the opening portion of the V-shaped cradle 18 and wedge surface 22B faces the rounded apex 20 of the V-shaped cradle 18. The video baton 30 includes a baton handle 33, a cable 34, a camera 35, a substantially circular side post 36 located on opposing sides of the baton handle 33, and a conduit 38 to convey optical information signals between the video camera and lighting member 35 and a display device, such as a TV or computer monitor depicted in FIGS. 24A and 26 below. The side post 36 detachably engages with the locking tab 22 to secure the video baton 30 within the sheath 12 and to allow its ready removal after performing a layrngoscopic procedure within a patient.

The sterilizeable sheath 12, though intended and packaged for single-use and then discarded after the removeable affixable video baton 30 is detached from the transparent sheath 12, may, in re-usable embodiments, be cleaned and either decontaminated to destroy pathogenic organism or autoclaved or subjected to other sterilization processes to destroy pathogenic and non-pathogenic organisms. Re-sterilizing processes may include autoclave steam processes, a mixture of autoclave steam and acid or other chemical fumes, gamma irradiation processes, and chemical sterilization processes, for example, ethylene oxide sterilization. In other circumstances, the sterilizable sheath 12 may be subjected to dry heat-based, moist-heat based, or chemical-based decontamination processes to destroy pathogenic organisms.

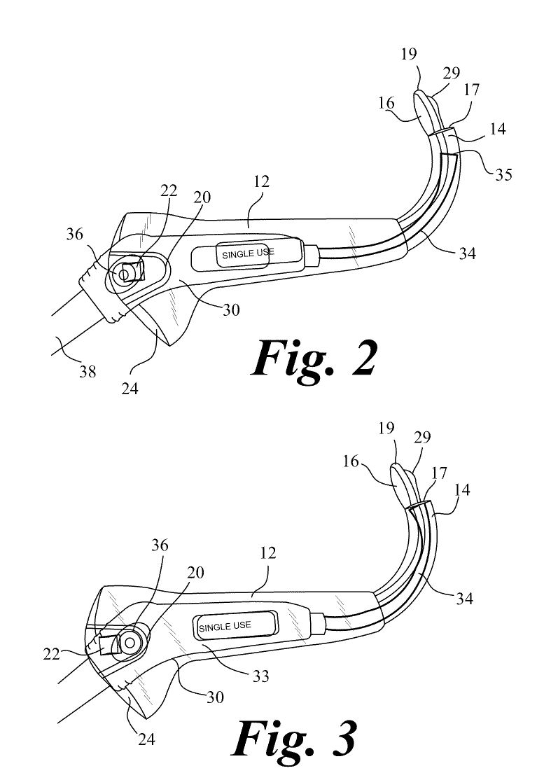

FIG. 2 depicts the detachable video baton 30 during insertion into the opening 24 of the sterilizeable sheath 12 in which the baton's side posts 36 slidably engage the side-securing tabs 22 and is shown to be transiting midway across the sloping surfaces of wedge 22A of the side-securing tabs 22. The video and lighting member 35 is shown in partial transition to the optically clear video window 17 and does not yet abut against it.

FIG. 3 depicts the finished insertion movement of the video baton 30 into the chamber 24 of the sheath 12 in which the baton's side posts 36 have transited across the tab's 22 sloping surfaces or wedges 22A and 22B and held in the rounded apex of a concave V-shaped cradle 18 and the inward slope 22B of the side-securing tab or lock 22. The camera 35 abuts against the optical window 17.

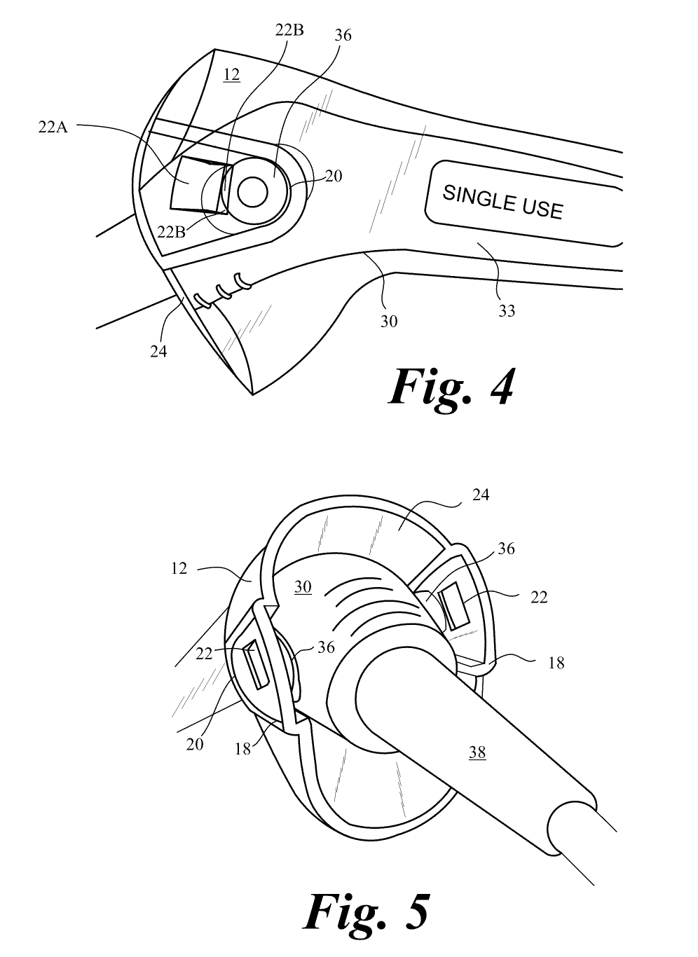

FIG. 4 depicts an enlargement of the baton side post 36 and sheath side-locking tab 22 depicted in FIG. 3. Here the side post 36 engages the smaller wedge surface 22B and the rounded apex 20 of the cradle 18 to securely hold the video baton 30 residing in the stat or sheath 12 while undergoing laryngoscope procedures.

FIG. 5 depicts the sheath chamber 24 with the video baton 30 completely engaged and secured within the V-shaped cradles 18 of the sheath 12. Locking tabs 22 are fully engaged with the posts 36 of the video baton 30.

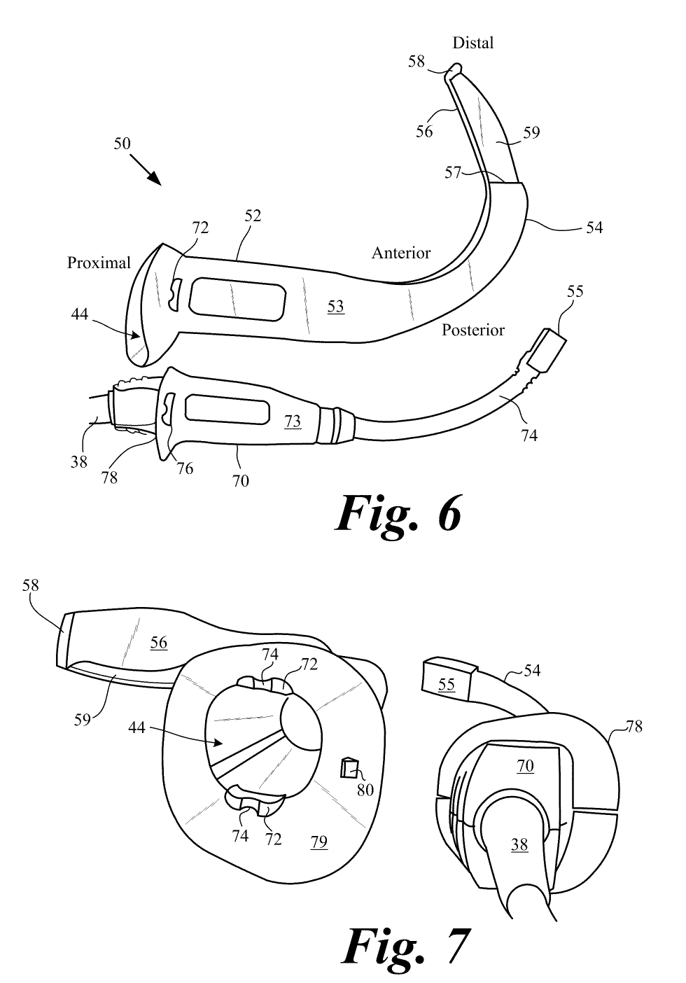

FIG. 6 depicts a substantially side view of a video laryngoscope system 50 having a single-use sterilizeable sheath 52 configured to receive a detachable video baton 70 with camera 55 and stabilized with the video baton's side ledges 76 and rearward lip 78 respectively engaged with the internal side supports 72 and rearward locking tab 80 of the sheath 52. The sterilizable sheath 52 includes a chamber 44 into which the video baton 70 may be inserted into and secured. The sterilizable sheath 52 may be transparent or opaque. The camera 55 may be brought into touchable contact with the optically clear window 57 of transparent sheath 52. The handle 73 of the baton 70 may be made to occupy the sheath handle chamber 53.

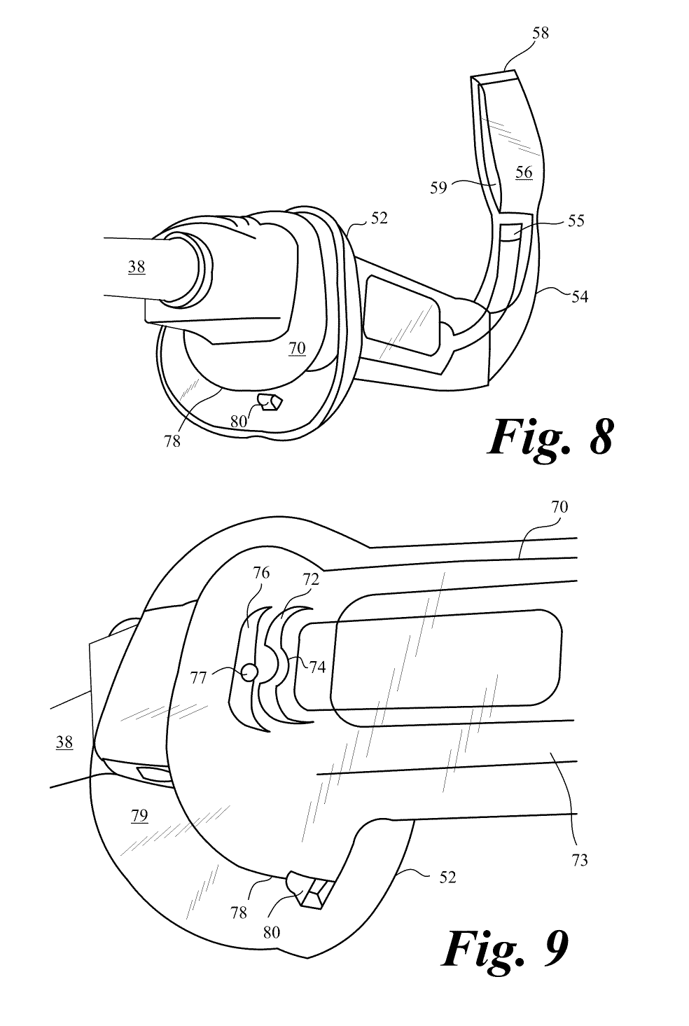

FIG. 7 depicts a substantially rear perspective view of the video laryngoscope system 50 showing the chamber 44 in which the internal side supports 72 are located on the walls of the baton handle chamber 53 that is continuous with a sheath brim 79 on which is mounted the rearward locking tab 80 to respectively engage the baton handle's 73 ledges 76 and lip 78.

FIG. 8 depicts a rearward perspective view of the detachable video baton 70 of FIG. 6 during insertion into the sterilizeable sheath 52. Here the locking tab 80 is not yet touchably engaged with the baton's 70 proximal lip 78.

FIG. 9 depicts a side perspective view of a portion of the detachable video baton 70 of FIG. 6 during insertion into the sterilizeable sheath 52. Here the locking tab 80 is not yet engaged with proximal lip 78 of baton 70. Similarly ledges 76 of baton 30 have not yet touchably engaged with the side supports 72 of sheath 52. In this side view, the ledges 76 of baton 30 are shown to have a curved shaped complimentary with side support 72 of sheath 52 in that side support 72 has a central cup-like concave portion 74 that receives a convex shape hemisphere ball or centering pin 77 extending from the side support 72 of baton 70.

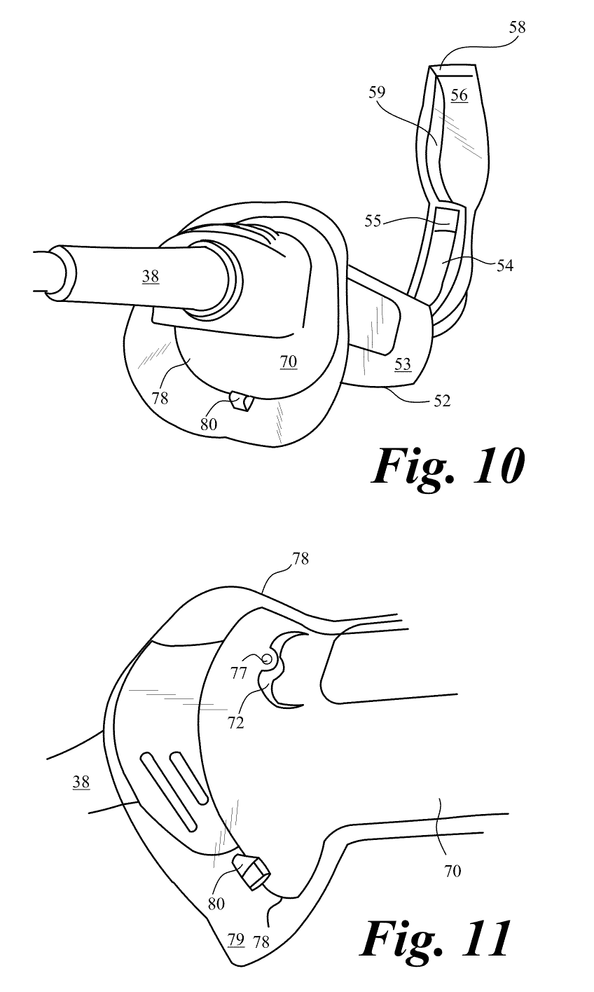

FIG. 10 depicts a rearward perspective view of the detachable video baton 70 of FIG. 6 inserted and secured into the sterilizeable sheath 52. Here the tab 80 of sheath 52 has flexed around to securely hold against the edge or lip 78 of baton 70.

FIG. 11 depicts a sideward perspective view of the portion of the detachable video baton 70 of FIG. 6 inserted and secured into the sterilizeable sheath 52. Here the tab 80 of sheath 52 has flexed around to securely hold against the edge 78 of baton 70 and the side supports 72 of sheath 52 engage with the surface of ledge or lip 76 and centering pin 77 of baton 70.

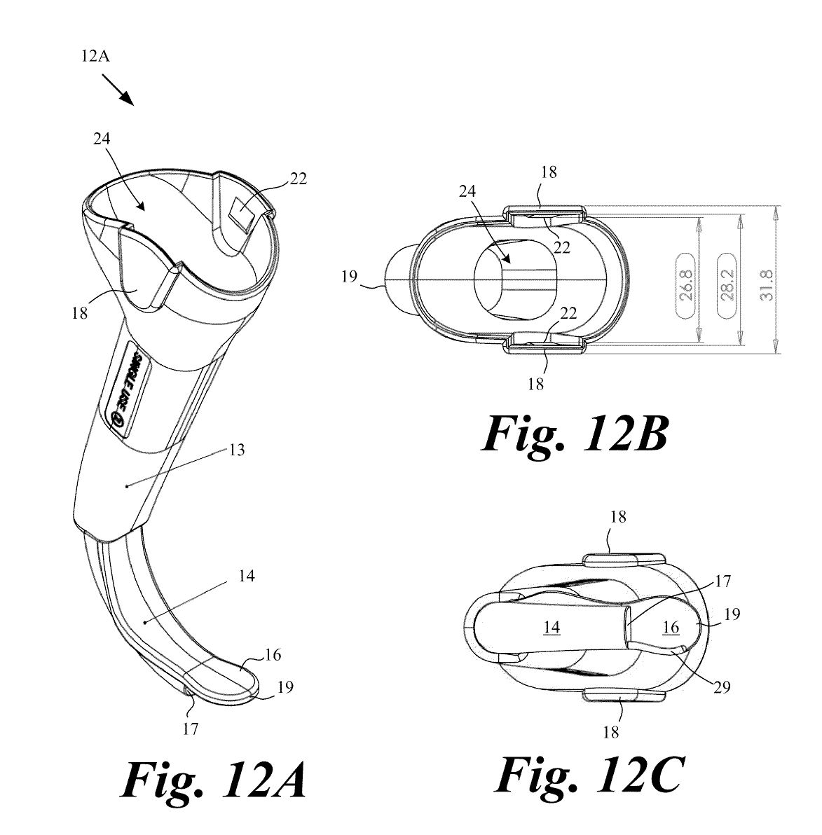

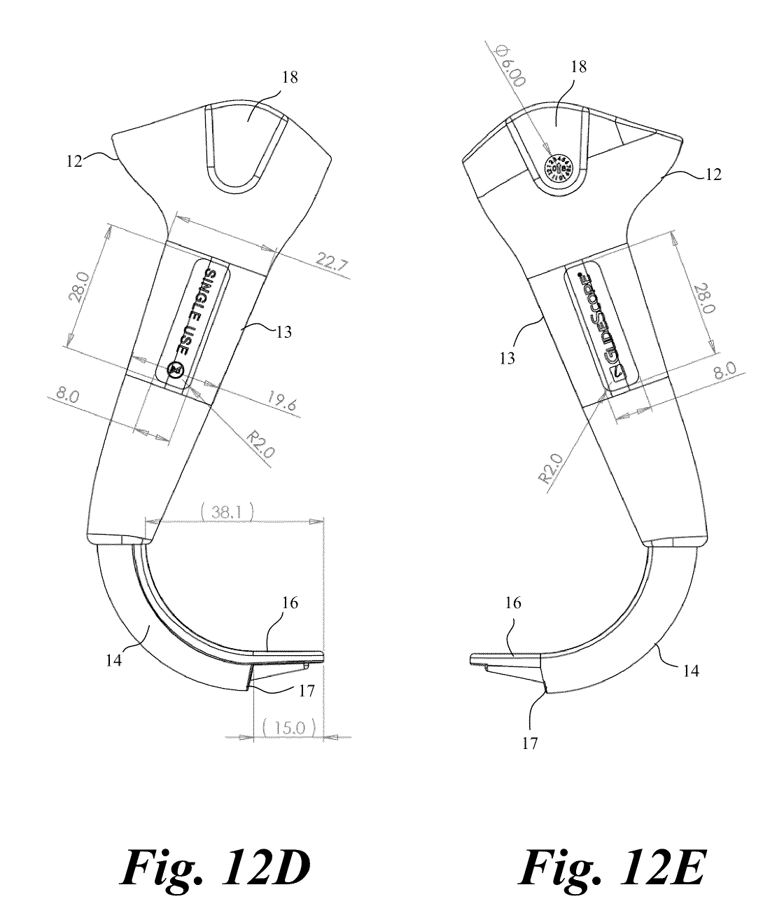

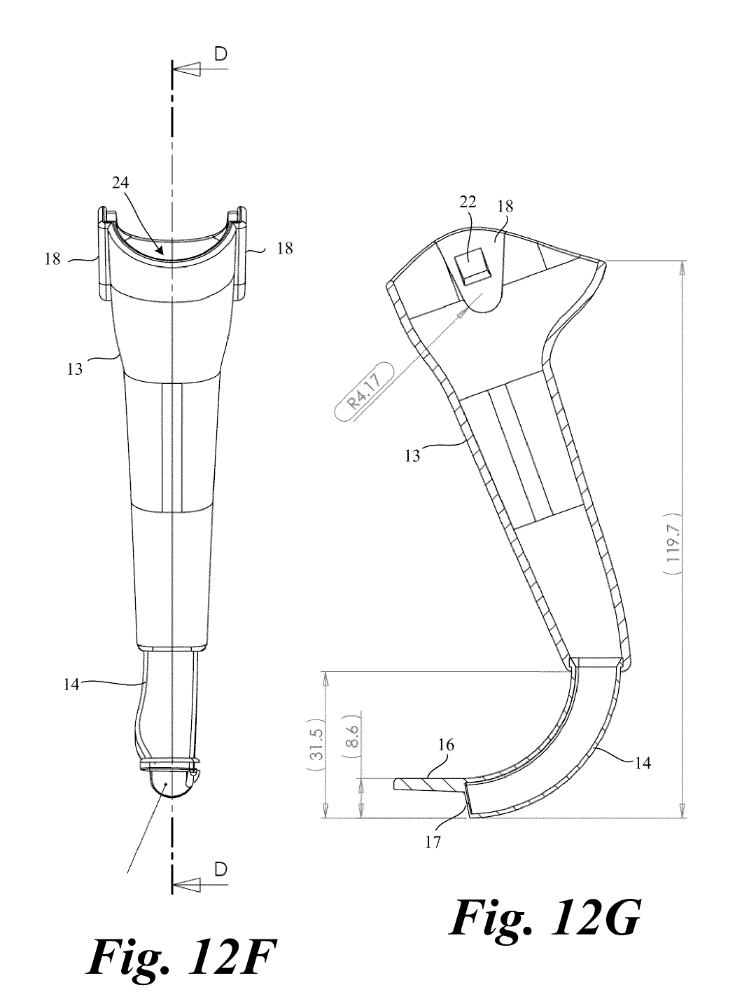

FIGS. 12A-G depict constructional plane and cross sectional views of an embodiment 12A of the sheath 12 depicted in FIG. 1. Top, bottom, left, right, perspective, front, and cross sectional views are depicted showing differences in the dimensions of distal blade portion 16A for a given sheath handle 13 configuration. The sheath 12A includes the chamber 24 into which the video baton 30 occupies. The handle 13 is over-molded onto the proximal blade portion 14 and distal blade portion 16 with a hermetic seal. The sheath 12A is manufactured substantially without flash, protruded gate marks, scratches, specks, bubbles, or weld lines in the area of the window 17. The sheath 12A is also substantially free of fingerprints, grease, dirt, or other contaminants. Particular embodiments of the sheath 12A may involve the plastic material being original, that is, not a re-grind or re-cycled plastic, and that plastic molding processes follow Sabic/GE Plastic Processing Guide for LEXAN HPS1 without the use of mold release agents. The 28 mm, 22.7 mm, 19.6 mm, 8.0 mm, 38.1 mm, 15 mm, and 2 mm dimensions depicted in FIG. 12D are exemplary. Similarly, the 119.7 mm, 22.7 mm, 31.5 mm, 8.6 mm and 4.17 mm dimensions depicted in FIG. 12G are exemplary.

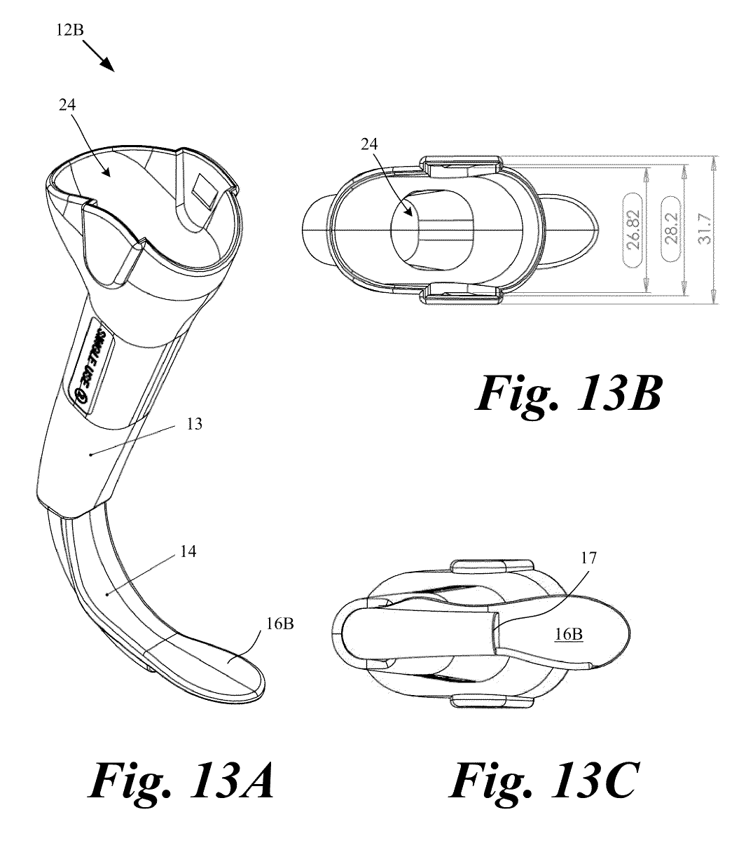

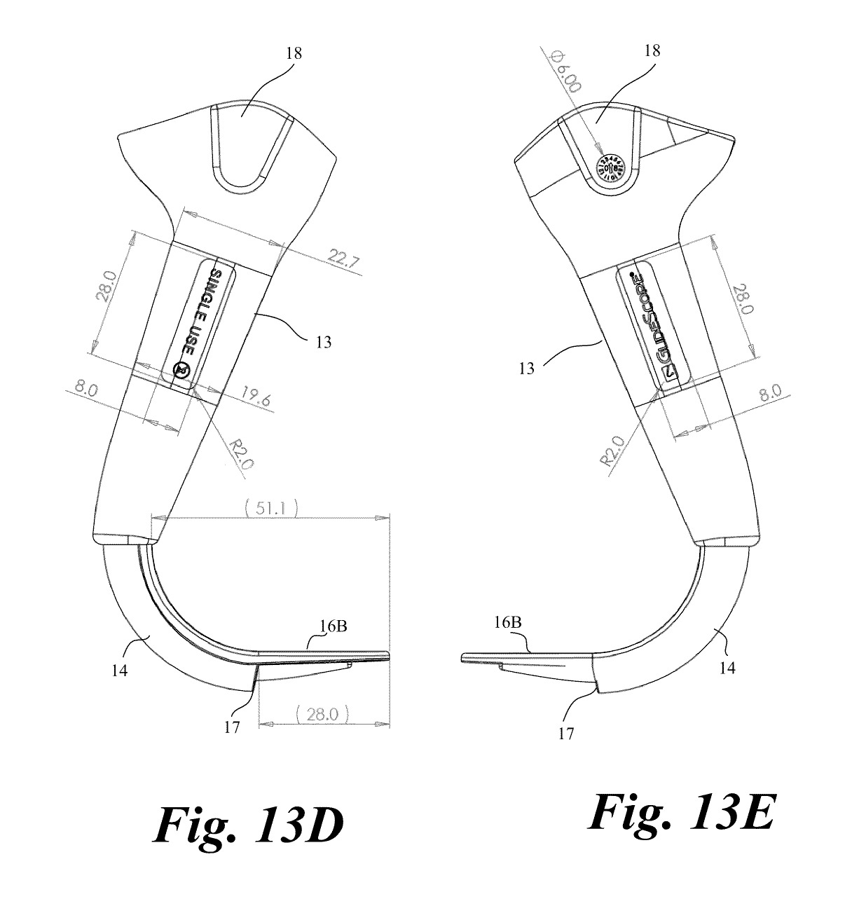

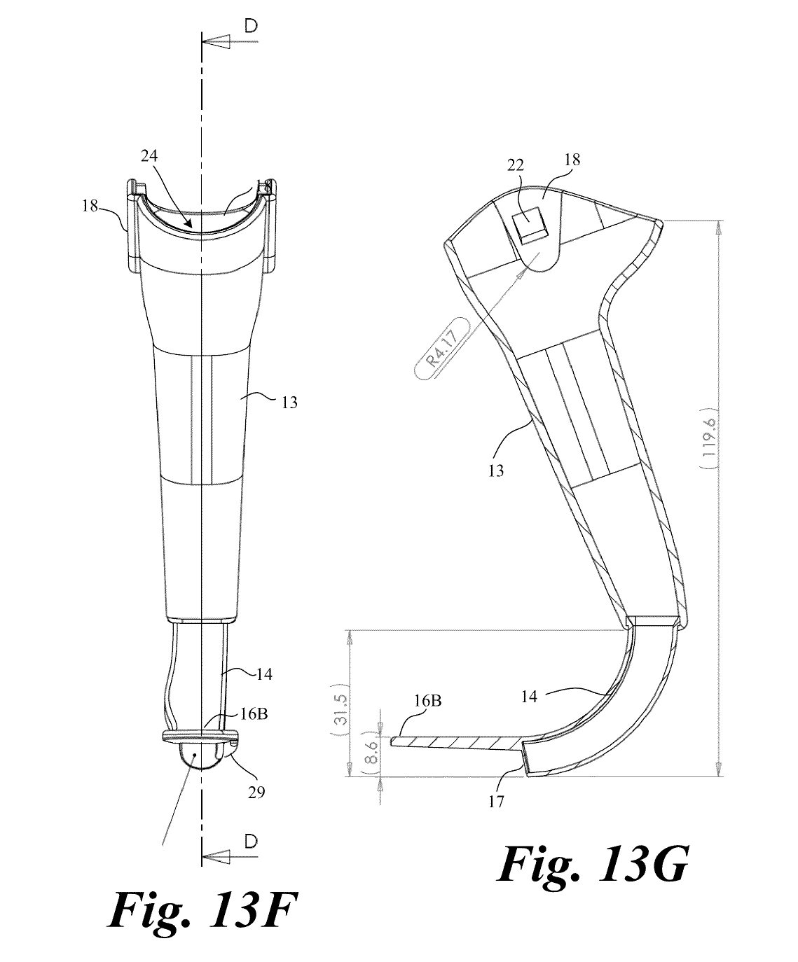

FIGS. 13A-G depict constructional plane and cross sectional views of another embodiment 12B of the sheath 12 depicted in FIG. 1. Top, bottom, left, right, perspective, front, and cross sectional views are depicted showing differences in the dimensions of distal blade portion 16B for a given handle 13 configuration. The sheath 12B includes the chamber 24 into which the video baton occupies. Manufacturing processes for the sheath 12B are substantially the same as for the sheath 12A of FIGS. 12A-G. Dimensions depicted in these figures are exemplary.

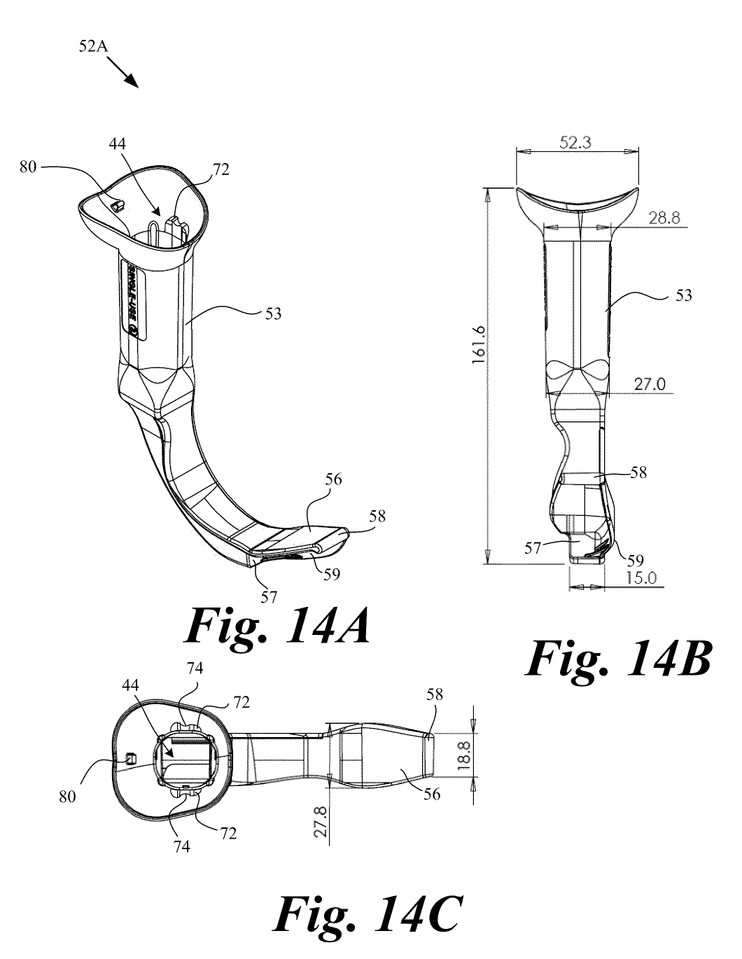

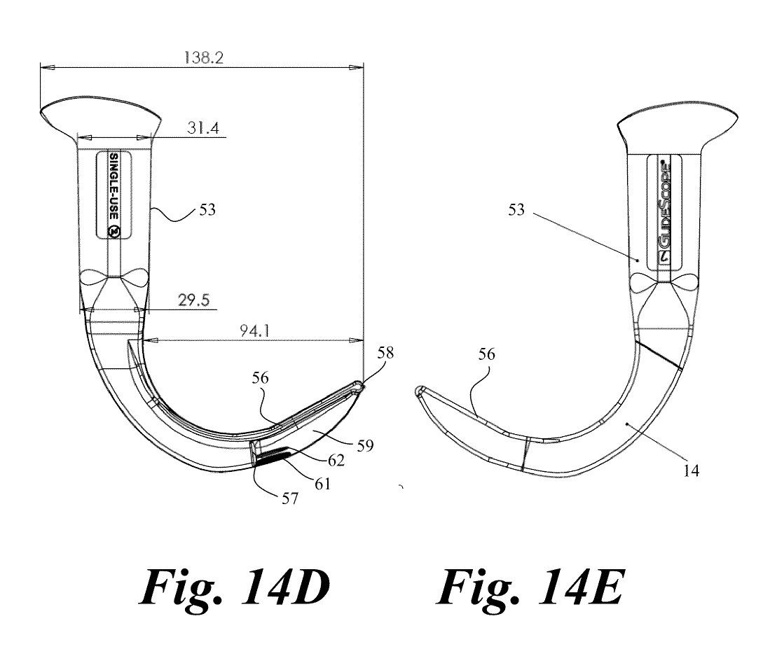

FIGS. 14A-E depict constructional plane and cross sectional views of an embodiment 52A of the sheath 52 depicted in FIG. 6. Top, bottom, left, right, perspective, and front, views are depicted showing differences in the tongue blade 56A dimensions for a given handle 53 configuration. The sheath 52A includes the chamber 44 into which the video baton 70 occupies. The handle 53 is ultrasonically welded onto the proximal blade portion 14 and distal blade portion 16 with a hermetic seal. The sheath 52A is manufactured substantially without flash, protruded gate marks, scratches, specks, bubbles, or ultrasonic weld lines in the area of the window 57. The sheath 52A is also substantially free of fingerprints, grease, dirt, or other contaminants. Particular embodiments of the sheath 52A may involve the plastic material being original, that is, not a re-grind or re-cycled plastic, and that plastic molding process follow Chevron Phillips Plastics Processing Guide for K-Resin SBC KRO3. Other particular embodiments of the sheath 52A are manufactured without the use of mold release agents. Braces 61,62 span across and secure flange 59 to the lip beneath the optical window 57. Dimensions depicted in these figures are exemplary.

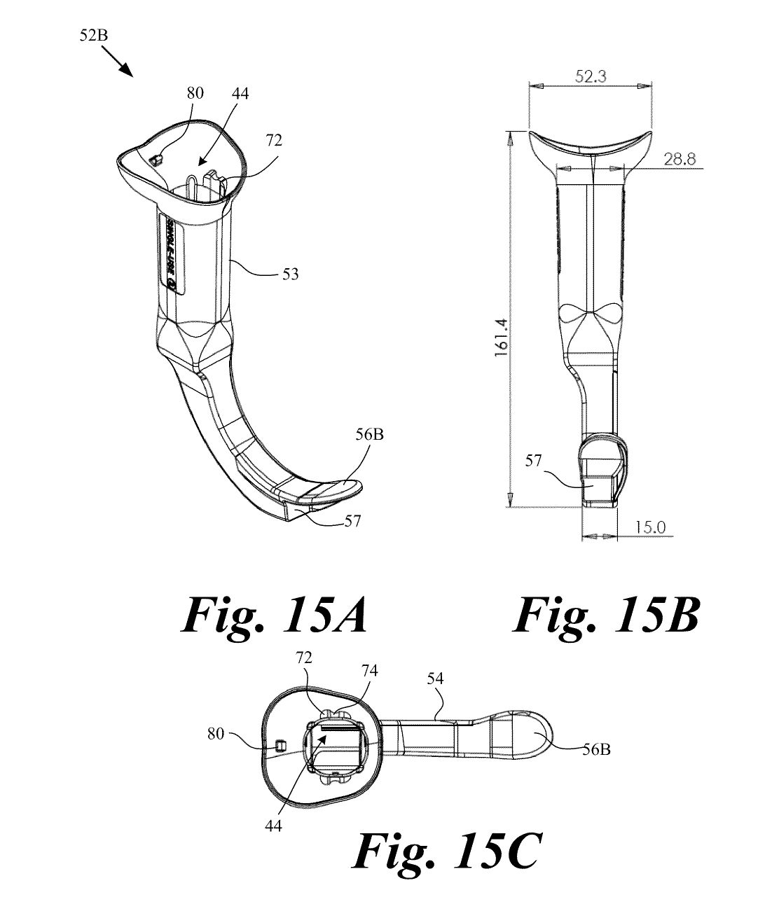

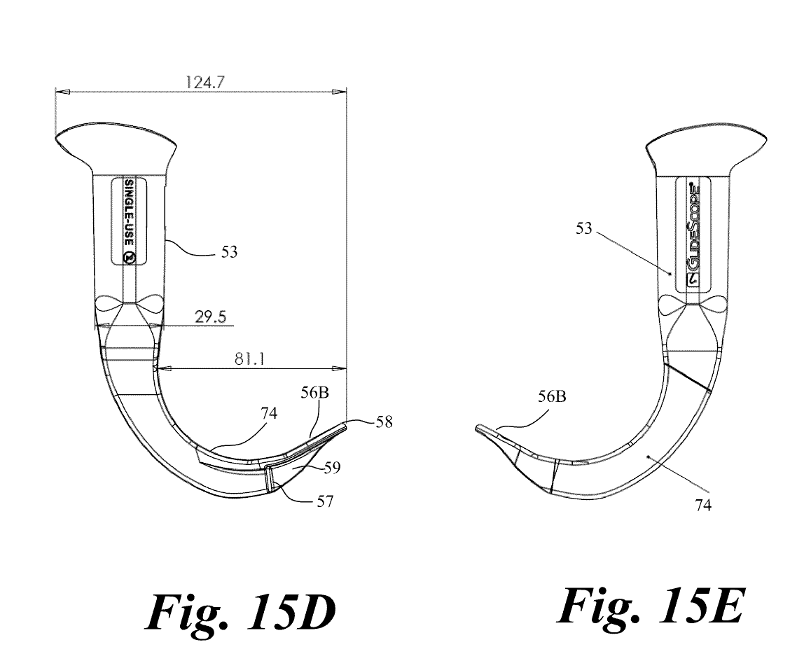

FIGS. 15A-E depict constructional plane and cross sectional views of another embodiment 52B of the sheath 52 depicted in FIG. 6. Top, bottom, left, right, perspective, and front, views are depicted showing differences in the dimensions of distal blade portion 56B for a given handle 53 configuration. The sheath 52B includes the chamber 44 into which the video baton 70 occupies. Manufacturing processes for the sheath 52B are substantially the same as for the sheath 52A of FIGS. 14A-E. Dimensions depicted in these figures are exemplary.

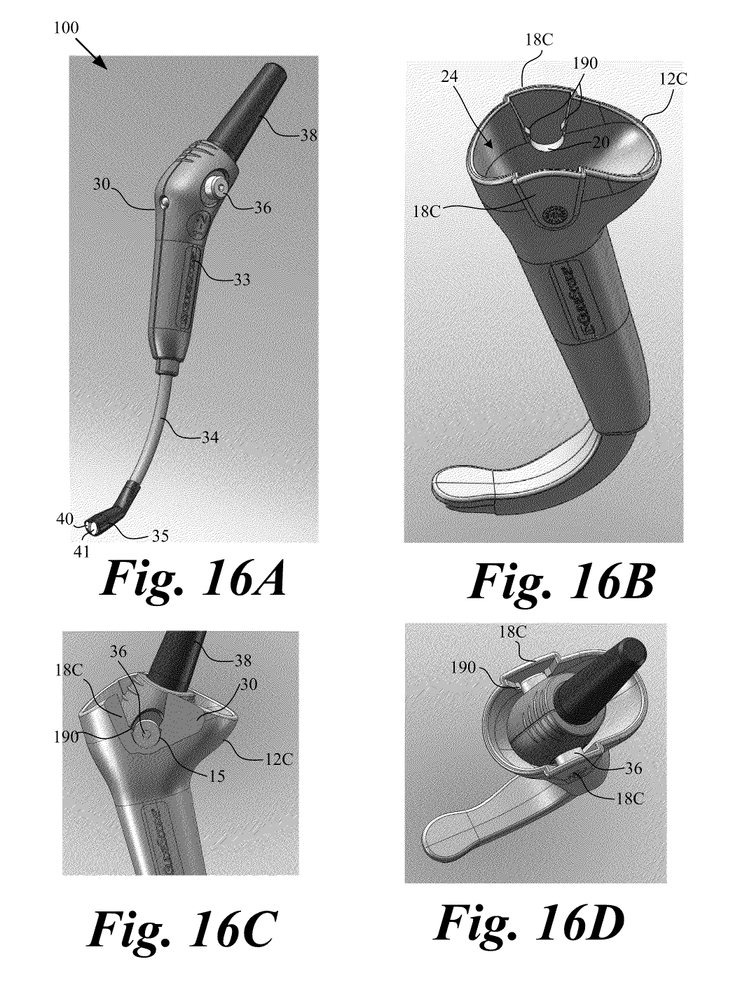

FIGS. 16A-D depict a series of substantially side and perspective views of a video laryngoscope system 100 having a single-use non-transparent sterilizeable sheath configured to receive a detachable video baton stabilized with the video baton's side posts that are engageable with opposing side-securing tabs 190 located extending from the edges of the V-shape cradle 18C of an alternate embodiment of the sheath 12 depicted in FIG. 1. A light source 40 and camera lens 41 are shown as part of the video camera and lighting member 35.

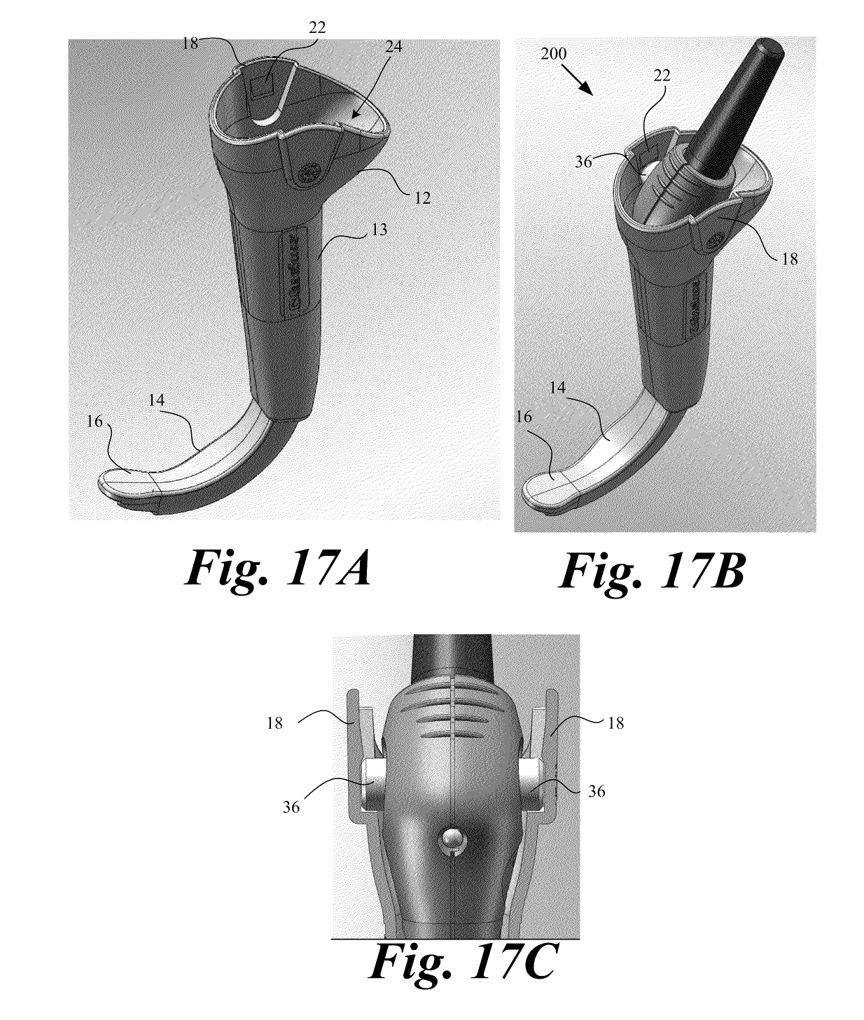

FIGS. 17A-C depict a series of substantially perspective views and a partial cross section view of the sheath in a video laryngoscope system 200 having a non-transparent sheath of as an alternated embodiment to the transparent sheath depicted in FIG. 1. Engagement of the post 36 in the rounded apex of the V-shaped cradle 18C and the wedge surface 22B is shown cross-section FIG. 17C.

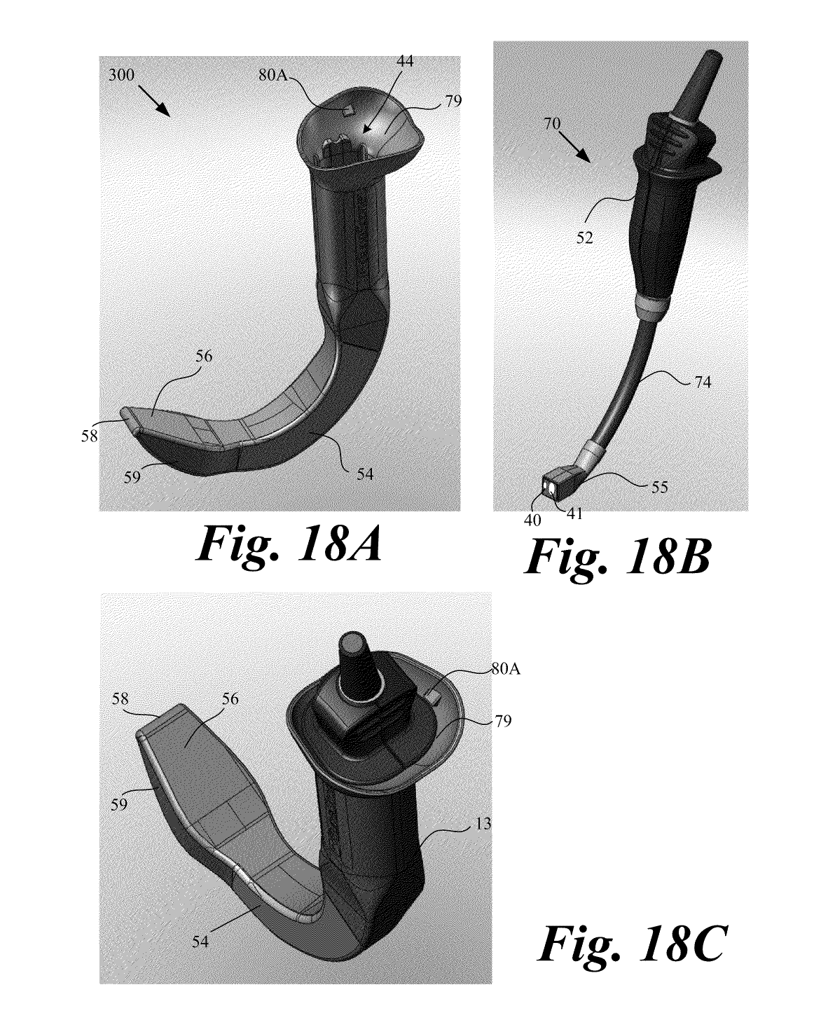

FIGS. 18A-C depict a substantially side perspective views of a video laryngoscope system 300 having a single-use non-transparent sterilizeable sheath configured to receive a detachable video baton 70 stabilized with the video baton's side ledges and rearward lip respectively engaged with the internal side supports and sideward locking tab 80A located on the brim 79 of an alternate embodiment of the sheath depicted in FIG. 6. The sideward locking tab 80A is approximately 90 degrees from the rearward locking tab 80 depicted in the sheath 52 of FIG. 6. The laryngoscope sheath 300 includes the chamber 44 into which the video baton 70 occupies. A light source 40 and camera lens 41 are shown as part of the camera 55.

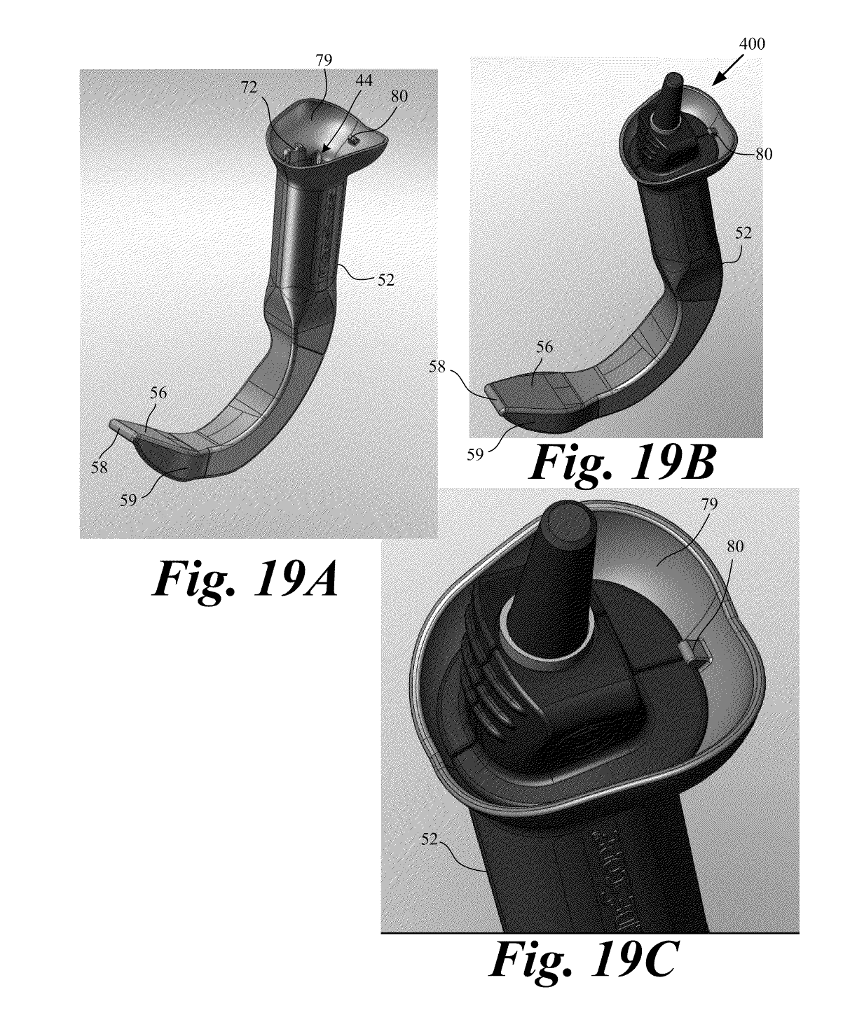

FIGS. 19A-C depicts substantially side perspective views of a video laryngoscope system 400 having a single-use non-transparent sterilizeable sheath 52 as an alternate embodiment of the transparent sheath 12 depicted in FIG. 1, the non-transparent sheath 52 configured to receive a detachable video baton stabilized with the video baton's side posts that are engageable with the side supports 72 and secured in place via engagement with tab 80 of the sheath 52. Chamber 44 is shown into which the video baton 70 occupies.

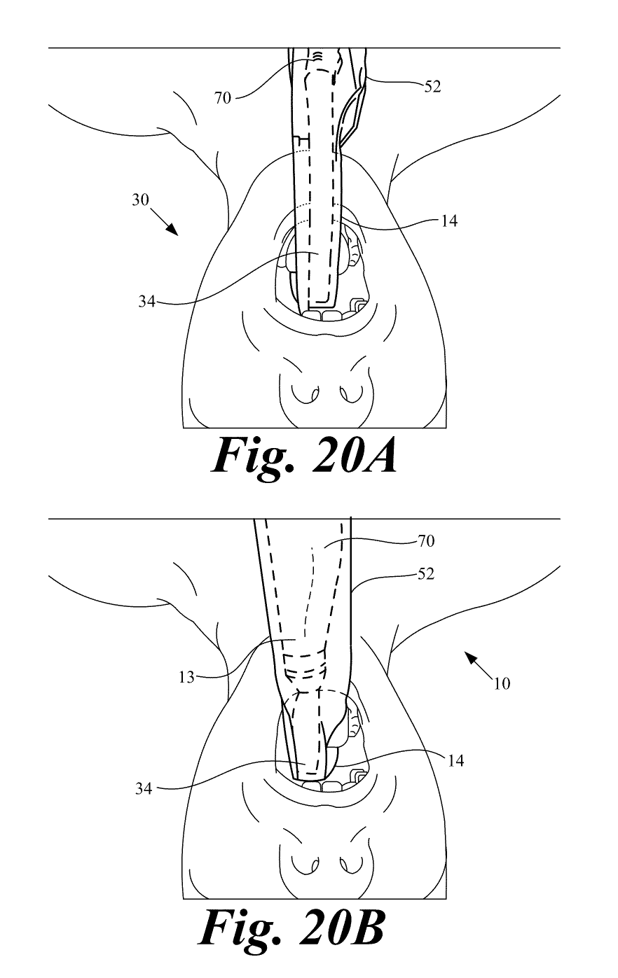

FIGS. 20A-B depict laryngoscope placement and advancement in a patient's mouth of laryngoscope 50 in which video baton 70 is detachably placed into and secured within the sheath 52. Other laryngoscope sheath-video baton embodiments may be similarly inserted and advanced in the patient's mouth, including laryngoscope 10, 50, 52A, 52B, 100, 200, 300, and 400. Laryngoscopes 5-400 may be positioned within the patient to engage the vallecula V via the respective distal tips of sheaths 10-53 to lift the epiglottis EPI by pressing the respective tips into the vallecula V and thereby expose the glottic aperture GA. Alternatively, the anterior surface of the distal blade portion 16, 16B, 56, 56B, and 58 may directly lift the epiglottis EPI to reveal the glottic aperture GA to lens view of video camera and lighting member 35.

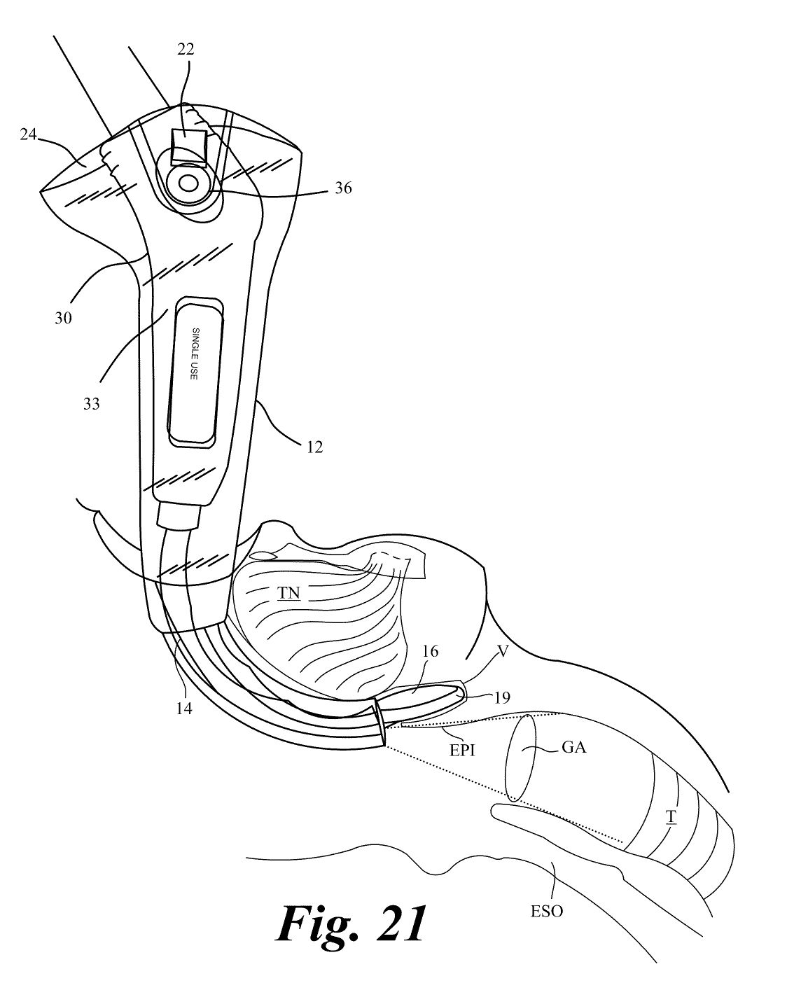

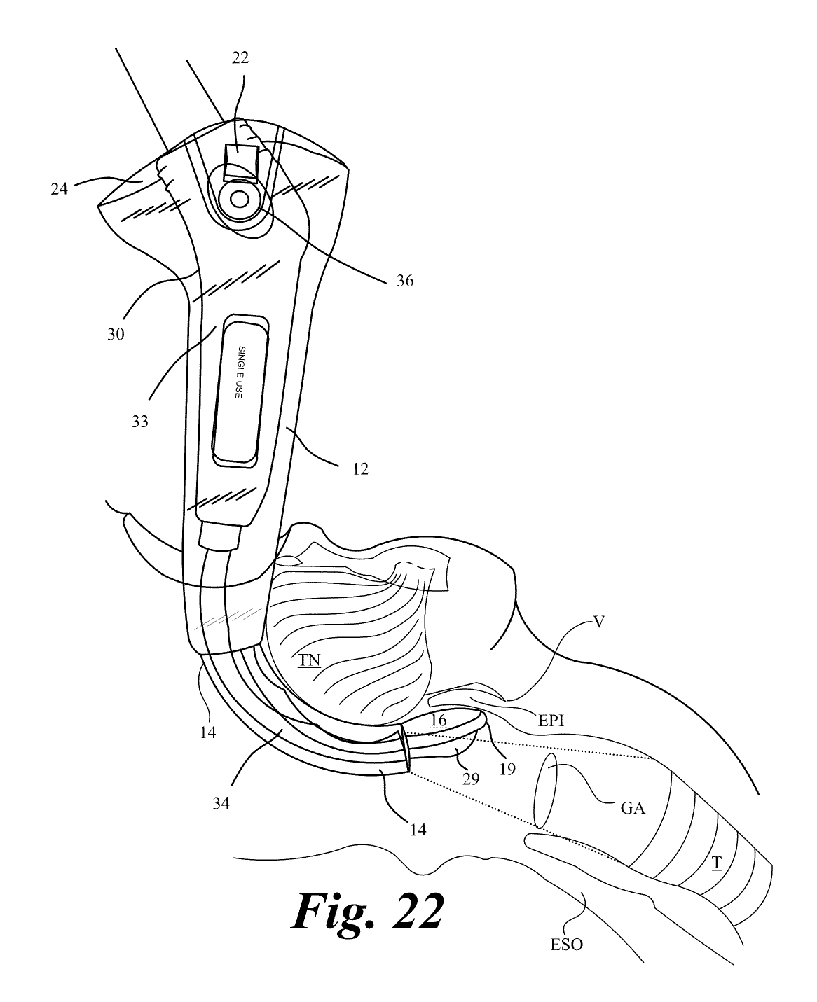

FIGS. 21 and 22 below depict side perspective view of laryngoscope 10 inserted into a patient shown in cross-section in which the glottic aperture GA is revealed by vallecula V engagement to swing up the epiglottis EPI or direct lifting of the epiglottis EPI. The anterior portion of the laryngoscope sheath 12 contacts the surface of the tongue TN. Beneath the trachea T is seen the esophagus ESO.

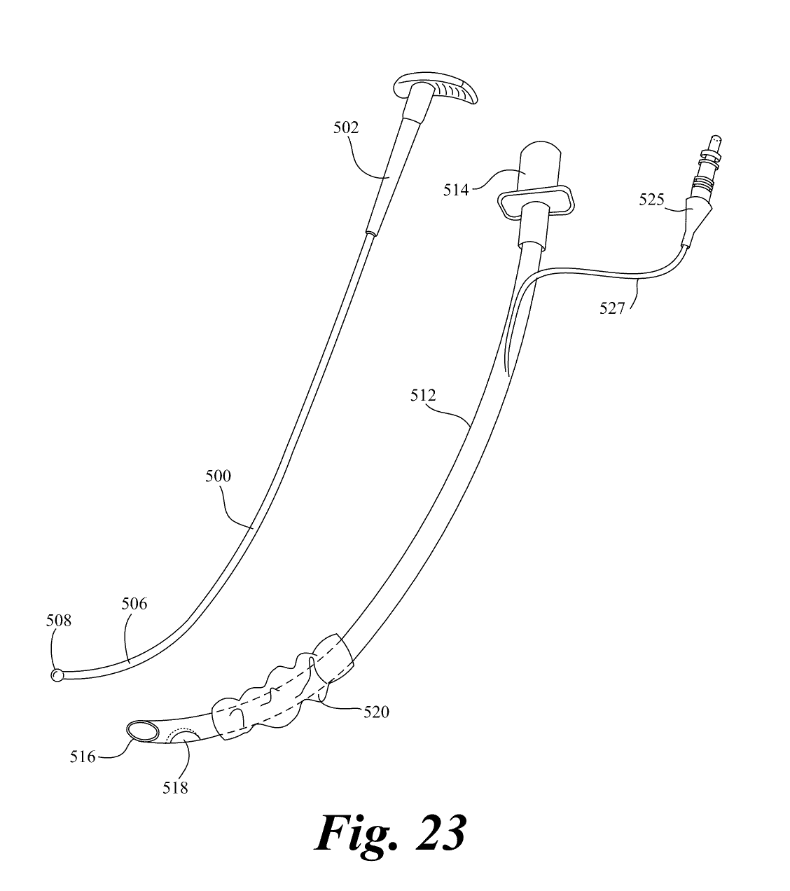

FIG. 21 depicts engagement of the vallecula V by the distal tip 19 of laryngoscope 12 sheath 10 equipped with video baton 30 to move the epiglottis EPI upward to reveal the glottic aperture GA. The epiglottis EPI normally obscures the glottic aperture GA. The tip 19 presses against the vallecula V to tense the hyo-epiglottic ligament (not shown) to pull the epiglottis upwards and expose the glottic aperture GA for passage of the ETT 512 shown in FIG. 23 below via stylet 500 manipulation described in FIGS. 25-27 below. The glottic aperture and other anatomical areas are illuminated (dotted lines) by the light source of the video camera and light source member 35.

FIG. 22 depicts direct lifting of the epiglottis by the tip or ridge 19 of laryngoscope sheath 12 to move the epiglottis EPI upward to reveal the glottic aperture GA. The laryngoscope sheath 12 is equipped with the video baton 30 in place. The video camera and lighting member 35 illuminates (dotted lines) the glottic aperture GA region for observation and passage of the endotracheal tube (“ETT”) 512 for placement within the trachea T.

FIG. 23 depicts a stylet 500 adjacent to an ETT 512. The ETT 512 includes a hose connector 514, a terminal aperture 516, a side aperture 518, a gas expandable balloon or cuff 520, and gas port 525 configured to receive a syringe (not shown) to convey gas or air through air tube 527 hydraulically connected with the cuff 520. The side aperture 518 is also known as a “Murphy's Eye”. The ETT 512 is shown to acquire a curvature amenable to following a trajectory to the glottic aperture GA by conforming to the curved shape of the stylet 500. The stylet 500 includes a handle 502, a rigid rod 504 extending from the handle and having a linear portion and a curved distal region 506 that terminates with a ball shaped end 508. The ball shaped end 508 is routed through the lumen of the ETT 512 and placed near the terminal aperture 516 or adjacent to the Murphy's Eye 518.

FIGS. 24A and B depict placement and advancement of the ETT via the stylet in upper airway of the patient using sheath and laryngoscope embodiment 50 depicted in FIGS. 6 and 10 in which the video baton 70 is secured within the sheath 52 by tab 80 engaged with the proximal lip 78 of video baton 70 more easily seen in FIG. 24B below.

FIG. 24A depicts a laryngoscope system 600 being deployed on a patient. The laryngoscope system 600 includes the sheath and laryngoscope embodiment 50 placed with the patient's upper airway for sending images from the video baton 70 to the monitor 610 via power and video cable 38. The monitor 610 includes a monitor control panel 624 to adjust images presented thereon. The ETT 512 loaded with stylet 500 is placed adjacent to the laryngoscope 50 and inserted into the patient's oral cavity under direct vision by the laryngoscope user.

FIG. 24B depicts advancement of the ETT 512 within the oropharynx of the patient under direct vision of the laryngoscope 50 user. The curved 506 region of stylet 500 is shown occupying the lumen of the ETT 512 with the stylet ball end 508 proximate to terminal aperture 516. The securing tab 80 of sheath 52 is engaged against the proximal lip 78 of the video baton 52.

FIG. 25 depicts a cross-sectional view of a patient's upper airway where the stylet 500 loaded ETT 512 is shown advancing into the upper airway adjacent to the blade portion 56 of laryngoscope 50. The ETT 512 and proximal blade is in contact with the patient's tongue TN. Tip 58 of laryngoscope 50 is shown directly lifting the patient's epiglottis EPI to reveal the glottic aperture GA. A light beam (dotted lines) emanating from the video camera 55 illuminates the glottic aperture GA, adjacent vocal cords VC, trachea 10, and immediate surroundings, and images thereof conveyed to the monitor 610 when the ETT 512 is later sufficiently advanced into camera view as shown in FIG. 26 below. The stylet 500 is advanced within the upper airway to place the ETT within camera view and in front of the glottic aperture GA. Beneath the trachea T is the esophagus ESO. The anterior portion of the laryngoscope sheath 52 contacts the surface of the tongue TN.

FIGS. 26-29 depict screen shot views of an intubation process to place ETT 512 within the trachea T. The screen shot views are captured by camera 55 and conveyed to the display of monitor 610 and viewed by the laryngoscope user to advance and place the ETT 512 into the trachea T.

FIG. 26 depicts a first monitor view 630 showing positioning of the ETT 512 in front of the glottic aperture GA for passage beyond the vocal cords VC into the trachea T. Flange 59 is in camera view on the upper left side and the ETT 512 is on the lower right side of the camera view as shown in first monitor view 630. The ball end 508 is shown adjacent to Murphy's Eye 518. The position of the vocal cords VC, trachea T, and arytenoids AR are depicted.

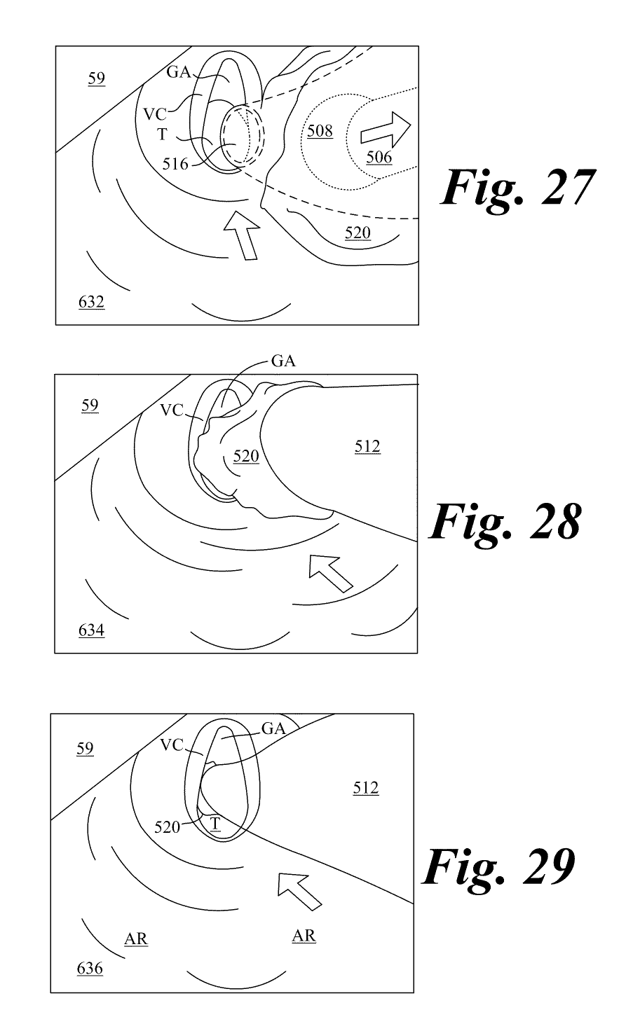

FIG. 27 depicts a second monitor view 632 showing advancement of the ETT 512 towards and just entering the glottic aperture GA with near simultaneous retraction of the stylet's 500 ball end 508 (see motion arrows). The cuff 520 advances into the larynx and the ball end 508, previously adjacent to the terminal aperture 516, now is moving proximally towards the user while the terminal aperture 516 is seen moving distally and just entering of the glottic aperture GA.

FIG. 28 depicts a third monitor view 634 showing advancement of the ETT 512 through the glottic aperture GA with the cuff 520 now entering the glottis aperture GA.

FIG. 29 depicts a fourth monitor view 636 showing advancement of the ETT's 512 cuff 520 past the glottic aperture GA and into the trachea T. The cuff 520 may now be inflated to expand and secure against the trachea walls by air injection via a syringe connected to the air port 525 and conveyed to the cuff 520 via air tube 527, shown in FIG. 23 above.

The system 600 can be employed for endotracheal intubation, laser-based surgical and biopsy procedures, and passage of ancillary equipment. Algorithms described below employ the laryngoscope 10, but may be suitably adapted to employ laryngoscopes 10, 50, 12A, 12B, 52A, 52B, 100, 200, 300, and 400. The intubation procedures can be adapted to place single or double lumen tubes. Similar procedures can be employed to, for example, remove a foreign body from the airway. Other laryngoscope procedures include directing a flexible laryngoscope, a bougie, or a bronchoscope and to guide ears-nose-throat (ENT) professionals, for example, to operate a jet ventilator or to perform biopsy and/or laser treatments for the patient.

The algorithms can be employed to enable the passage and control of a number of tools useful for surgery and procedures in and around the airway such as simultaneous visualization and conduct of surgical laser operating systems, electro-surgical operating batons, surgical biopsy instruments, surgical suction devices, jet ventilation systems for transglottic ventilation during laryngeal and airway surgery, double lumen endotracheal tubes commonly used for lung separation during surgical procedures on the thoracic structures, flexible bronchoscopes and gastroscopes, intubating bougie devices, Transesophageal Echo probes, and nasogastric tubes.

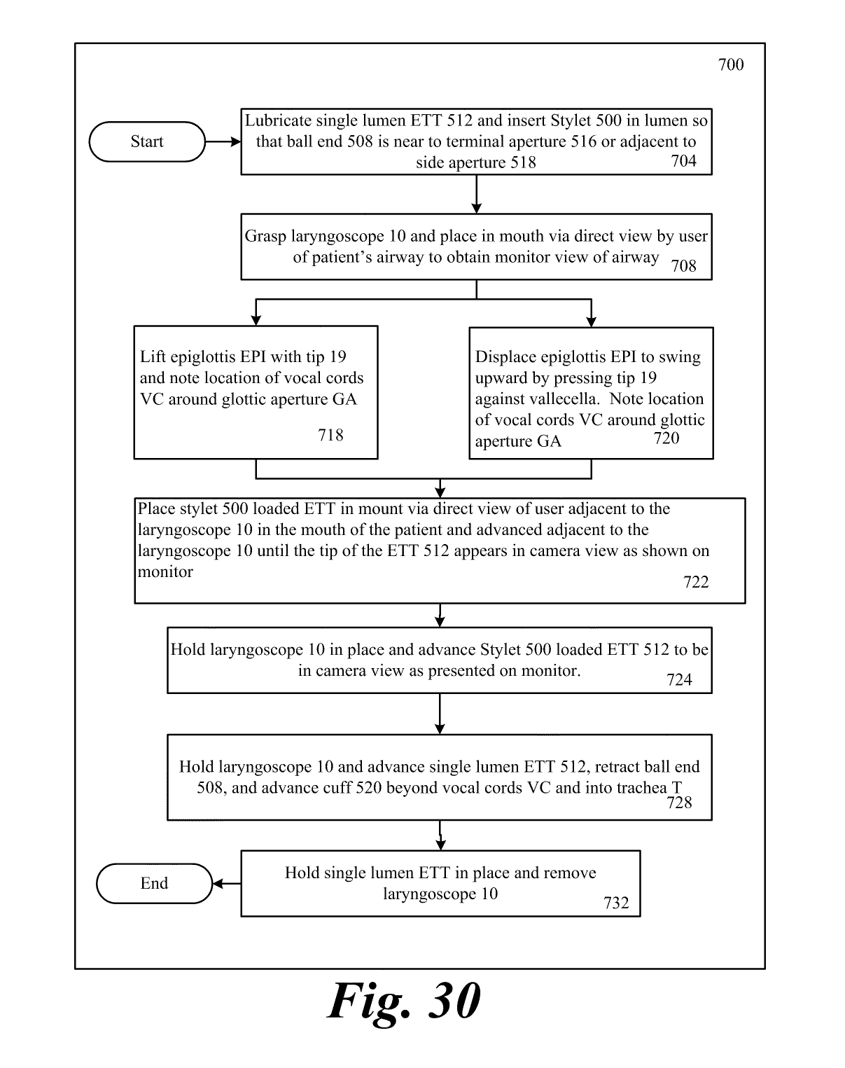

FIG. 30 depicts an intubation algorithm 700 for endotracheal intubation of a single lumen tube using an ETT 512 loaded with an accessory stylet 500 in which the epiglottis EPI is pivoted upwards by direct lifting or by engaging the vallecula V. Beginning at process block 704, a single lumen ETT 512 is lubricated and the Stylet 500 is inserted within ETT's 512 lumen so that the stylet ball end 508 is placed near the terminal aperture 516 or adjacent to the side aperture or Murphy's Eye 518. At process block 708, the laryngoscope 10 is grasped by the user's left hand and placed in the patient's mouth by direct vision or view of the user. Epiglottis displacement then can occur by two alternative processes. At process block 718, the epiglottis EPI is lifted with the tip 19 to reveal the glottic aperture GA. The location of vocal cords VC are noted by the user viewing images presented on the monitor 610. Alternatively, at process block 720, the epiglottis EPI is swung upwards or displaced upwards by pressing tip 19 against the patient's vallecula V. The location of the glottic aperture GA and vocal cords VC are similarly noted by the user's viewing the images presented on the monitor 610. At process block 722, the Stylet 500 loaded with the ETT 512 is inserted into the mouth under direct vision and advanced adjacent to the laryngoscope until the tip of the single lumen ETT 512 is visible on the monitor 610. At process block 724, while holding the laryngoscope 10 in place, the ETT 512 advanced into camera view as presented on the monitor 610. Thereafter, at process block 728, while holding the laryngoscope 10 in place, the single lumen ETT 512 is advanced distally towards and through the glottic aperture GA, beyond the vocal cords VC while the ball end 508 of the sytlet 500 is retracted proximally towards the user. The ETT 512 is sufficiently advanced distally to place the cuff 520 beyond the vocal cords VC and into the trachea T. Thereafter, algorithm 700 is completed by holding the advanced single lumen ETT 512 in place and the laryngoscope 10 is removed. Algorithm 700 can be similarly used in system 600 by employing the laryngoscope devices 50, 12A, 12B, 52A, 52B, 100, 200, 300, and 400.

An embodiment of the above includes a GlideScope® video enabled ENT laryngoscope substantially similar to the video-based sheath laryngoscope devices 10, 50, 12A, 12B, 52A, 52B, 100, 200, 300, and 400 as discussed above. The transparent and non-transparent sheaths or stats employed in the laryngoscope systems provide for tongue lifting and may be configured to provide an HDTV Video Bronchoscope amenable to rigorous disinfection procedures, a lighting source, and ancillary tools are delivered via open channels or around the body of the sheaths using a free hand.

There are several advantages to the GlideScope® video enabled ENT laryngoscope over existing devices: The one-time use of sterilized sheaths addresses the issue of BSE and Adult CJV in those jurisdictions where regulation requires disposable parts because no parts are reused in direct patient contact. The shape and sizes of the disposable sheaths of the system embodiments described above allows smaller forces to be used to gain access to the anatomical features reducing potential injury to the patient. The design allows the ENT surgeon a variety of different configurations based on the needs of the procedure being performed, equipment available and personal preferences. Disposable see-through or clear plastic sheaths may be adapted to the various embodiments described herein to provide efficient execution of laryngoscopic procedures between patients.

Other embodiments provide for a jet ventilation channel or conduit in the disposable sheaths so that jet ventilation is capable of being aimed approximately within 3-4 mm of the conduit. This renders a steady aim to be confidently established by an attending anesthesiologist. The steady aim allows the easy observation of the direction of the positive pressure discharge to assure that high-pressure gas does not enter the tissues but rather entrains air to ventilate the trachea and minimize pressure buildup.

The GlideScope® laryngoscope system provides for a video enabled laryngoscope that conveys visual confirmation of airway anatomy during airway procedures. Alternate embodiments provide for a disposable shell assembly to sheath the video apparatus and to provide a disposable option. The disposable design strategy provides effective cover for the video system. The electronic package may be fully immersible for cleaning and have all of the features of the regular video laryngoscope system so that its adoption into the medical theater does not require additional training procedures. The disposable shell assembly may be configured to have structural strength and durability to withstand sanitizing procedures.

The utility of a disposable shell or sheath option is that it provides the possibility of having a number of blade options suited to differing applications and clinical tasks. The range includes obesity, pediatrics, persons of small stature, normal adults, training designs, and neonatal designs. Thus with one master unit the shell size may be selected to suit the clinical situation.

Other embodiments may include a channel or channels passing into the larynx area that may have a number of supportive channels dedicated from time to time to differing functions. The electronic assembly may comprise a rigid or flexible wire lead to a camera which has a heated lens, a light emitting diode (LED) lighting array, and a charge coupled device (CCD), or a complementary metal-oxide-semiconductor (CMOS) digital video camera for real time video monitoring of the airway for intubation and diagnosis.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. An apparatus comprising:
a sheath having a handle with at least one retaining clasp, a blade having a window on the posterior side directed distally, and a chamber traversing through the sheath,
wherein a video baton upon insertion into the chamber places a sensor of the video baton adjacent to the window and the video baton is secured inside the sheath by the at least one retaining clasp.

2. The apparatus of claim 1, wherein the at least one retaining clasp includes an adjoining tapered wedge engageable with the surface of the exterior of the video baton.

3. The apparatus of claim 1, wherein the video baton includes a light source.

4. The apparatus of claim 1, wherein the video baton is removable from the chamber.

5. The apparatus of claim 1, wherein the laryngoscope housing comprises material amenable to undergo at least one of a cleaning process, a decontamination process, and a sterilization process.

6. The apparatus of claim 1, wherein the at least one retaining clasp is configured to break upon removal of the video baton from the chamber.

7. An apparatus comprising:
a sheath configured for insertion in the oropharynx of a patient, the sheath having a handle, a blade having a ridge distal to the handle, at least one retaining clasp, and a chamber traversing through the handle, beneath the blade, and having a window located near the distal end of the blade; and
a video member configured for insertion into the chamber,
wherein upon insertion of the video member the lens of the video member is placed adjacent to the window and is secured by the at least one retaining clasp engaging with the exterior of the video member and the ridge is configured to press against the vallecula of the patient to displace the epiglottis upward to reveal the glottic aperture.

8. The apparatus of claim 7, wherein the at least one retaining clasp includes adjoining tapered wedge engageable with the surface of the exterior of the video member.

9. The apparatus of claim 7, wherein the at least one retaining clasp is configured to break upon removal of the video member from the chamber

10. An apparatus comprising:
a sheath configured for insertion in the oropharynx of a patient, the sheath having a handle, a blade having a ridge distal to the handle, at least one retaining clasp, and a chamber traversing through the handle, beneath the blade, and having a window located near the distal end of the blade; and
a video member configured for insertion into the chamber,
wherein upon insertion of the video member the lens of the video member is placed adjacent to the window and is secured by the at least one retaining clasp engaging with the exterior of the video member and the ridge is configured to lift the epiglottis upward to reveal the glottic aperture.

11. The apparatus of claim 10, wherein the at least one retaining clasp includes adjoining tapered wedge engageable with the surface of the exterior of the video member.

12. The apparatus of claim 10, wherein the at least one retaining clasp is configured to break upon removal of the video member from the chamber.

13. A system comprising:
a sheath configured for insertion in the oropharynx of a patient, the sheath having a handle, a blade having a ridge distal to the handle, at least one retaining clasp, and a chamber traversing through the handle, beneath the blade, and having a window located near the distal end of the blade; and
a video member configured for insertion into the chamber; and
a monitor in signal communication with the video member,
wherein upon insertion of the video member the lens of the video member is placed adjacent to the window and is secured by the at least one retaining clasp engaging with the exterior of the video member, images are conveyed to the monitor, and the ridge is configured to do at least one of displacing the epiglottis of the patient upwards by pressing against the valeculla or to lift the epiglottis upwards to reveal the glottic aperture.

14. The system of claim 14, wherein the at least one retaining clasp is configured to break upon removal of the video member from the chamber.

15. A method comprising:
placing a sheath covered laryngoscope having a video camera in the oropharynx of a patient;
placing an endotracheal tube adjacent to the sheath covered laryngoscope in the oropharynx of the patient;
obtaining monitor-displayed images of the oropharynx, and based on the monitor displayed images:
displacing the endotracheal tube by the distal tip of the laryngoscope to reveal the glottic aperture; and
advancing the ETT through the glottic aperture, beyond the vocal cords, and into the trachea.

16. The method of claim 15, wherein displacing the epiglottis includes pressing the distal tip against the patient's vallecula to swing the epiglottis upwards.

17. The method of claim 15, wherein displacing the epiglottis includes lifting the epiglottis upwards by the distal tip.

18. The method of claim 15, wherein placing the endotracheal tube further comprises inserting a stylet in a lumen of the endotracheal tube.

19. The method of claim 18, wherein advancing the endotracheal tube includes retracting the stylet proximally towards the user while simultaneously advancing the endotracheal tube distally towards the glottic aperture.

20. A laryngoscope comprising:
a sheath further comprising;
an interior chamber defined by the sheath;
a sheath handle portion having at least one retaining clasp and defining an opening to the interior chamber;
a sheath blade portion for insertion into the mouth having a distal portion terminating with a distal tip, a proximal portion, an anterior side for controlling a tongue and engaging an epiglottis, and a window on a posterior side directed toward the distal tip; and
a video baton having a camera portion in communication with a baton handle portion; the video baton configured to fit within the interior chamber such that the camera portion is adjacent to the window and the baton handle portion is surrounded by the sheath handle portion when the at least one retaining clasp engages the video baton.
